(19) United States
(12) Patent Application Publication
(10) Pub. No.: US 2009/0182384 A1
(43) Pub. Date: Jul. 16, 2009

(54) MATERIAL COMBINATIONS FOR MEDICAL DEVICE IMPLANTS

(75) Inventors: Bryan Scott Wilcox, Collierville, TN (US); Rodney Ray Ballard, Lakeland, TN (US); Eric Daniel Densford, West Memphis, AR (US); James Michael Mirda, Cordova, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/013,838

(22) Filed: Jan. 14, 2008

(51) Int. Cl. A61B 17/86

(52) U.S. Cl. 606/305

(57) ABSTRACT

The present application is directed to embodiments of a pedicle screw assembly to position an elongated member within a patient. The assembly may include a receiver with a channel sized to receive the elongated member and a chamber. The assembly may also include an anchor with a head sized to fit within the chamber. The assembly may include a compression member sized to fit within the chamber and include a first side that contacts against the head and a second side that contacts against the receiver. At least one of the receiver, anchor, and compression member may be constructed of a first material, and at least one of the receiver, anchor, and compression member may be constructed of a second material. The first and second materials may include different moduli of elasticity to prevent deformation of the assembly.

### BACKGROUND

The present application is directed to a pedicle screw assembly and, more specifically to a pedicle screw assembly with elements constructed of different materials.

Various conditions may lead to damage of vertebral members and/or intervertebral discs. The damage may result from a variety of causes including a specific event such as trauma, a degenerative condition, a tumor, or infection. Damage to the intervertebral discs and vertebral members can lead to pain, neurological deficit, and/or loss of motion. Elongated members, such as but not limited to rods, bars, and plates, may extend along the spine to redistribute stresses and/or restore proper alignment of the vertebral members. The elongated members may be substantially straight, or include a curved configuration to conform to the curvature of the spine.

One or more pedicle screw assemblies attach the elongated members to the vertebral members. The assemblies are usually connected to the vertebral members at points along the spine where the elongated members are to be located. The assemblies should securely connect with the elongated members and provide a strong anchor for maintaining the position of the elongated member. The connection with the elongated member often proves difficult because of the stresses imposed to restore proper alignment of the vertebral members.

The assemblies should be constructed of materials with sufficient strength to withstand the stress induced by the spinal realignment. However, the assemblies are often bulky, and the materials used may interfere with magnetic resonance imaging, as well as impose dangers on the patient.

### SUMMARY

The present application is directed to embodiments of a pedicle screw assembly to position an elongated member within a patient. The assembly may include a receiver with a channel sized to receive the elongated member and a chamber. The assembly may also include an anchor with a head sized to fit within the chamber. The assembly may include a compression member sized to fit within the chamber and include a first side that contacts against the head and a second side that contacts against the receiver. At least one of the receiver, anchor, and compression member may be constructed of a first material, and at least one of the receiver, anchor, and compression member may be constructed of a second material. The first and second materials may include different moduli of elasticity to prevent deformation of the assembly.

### BRIEF DESCRIPTION OF THE DRAWINGS

FIG. 1 is a schematic diagram of a pedicle screw assembly according to one embodiment.

FIG. 2 is a perspective view of a pedicle screw assembly with an elongated member according to one embodiment.

FIG. 3 is a section view cut along line III-III of FIG. 2.

FIG. 4 is a perspective view of a receiver according to one embodiment.

FIG. 5 is a section view of a pedicle screw assembly according to one embodiment.

FIG. 6 is a schematic front view of a receiver according to one embodiment.

FIG. 7 is a schematic front view of a receiver according to one embodiment.

FIG. 8 is a schematic side view of a receiver according to one embodiment.

FIG. 9 is a schematic front view of a receiver according to one embodiment.

FIG. 10 is a schematic front view of a receiver according to one embodiment.

FIG. 11 is a schematic front view of a receiver according to one embodiment.

FIG. 12 is a schematic front view of a receiver according to one embodiment.

FIG. 13 is a section view of a receiver according to one embodiment.

FIG. 14 is a section view of a receiver according to one embodiment.

FIG. 15 is a section view of a compression member according to one embodiment.

FIG. 16 is an exploded perspective view of a receiver according to one embodiment.

FIG. 17 is a section view of a pedicle screw assembly with an elongated member according to one embodiment.

FIG. 18 is an exploded side view of a pedicle screw assembly according to one embodiment.

### DETAILED DESCRIPTION

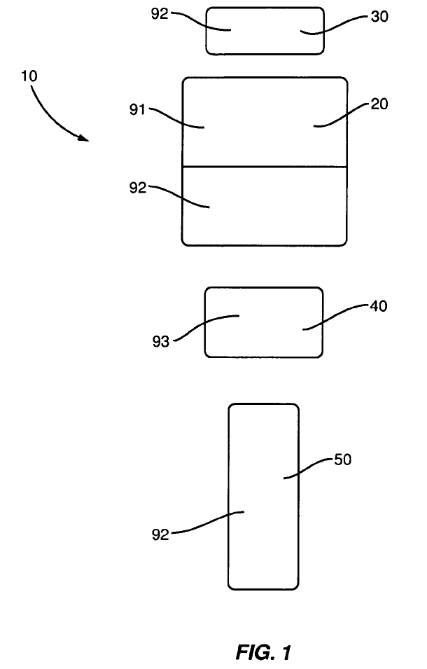

The present application is directed to embodiments of a pedicle screw assembly 10. FIG. 1 illustrates one embodiment of an assembly 10 constructed of elements that include a receiver 20, set screw 30, compression member 40, and a bone anchor 50. At least one of the elements is constructed in whole or in part from different materials than the other elements. FIG. 1 includes an embodiment with the receiver 20 constructed of first and second materials 91, 92, the set screw 30 and anchor 50 constructed of the second material 92, and the compression member 40 constructed of a third material 93. The different materials each include different moduli of elasticity to prevent deformation of the assembly 10.

The number of elements of the assembly 10 constructed of the first and second materials may vary. In one embodiment, only one element is constructed of the first material, with the other elements being constructed of the second material. In another embodiment, multiple elements are constructed of the first material and multiple elements are constructed of the second material. In one embodiment, one or more of the elements is constructed of a third material. Likewise, the number of elements constructed of at least two different materials may vary.

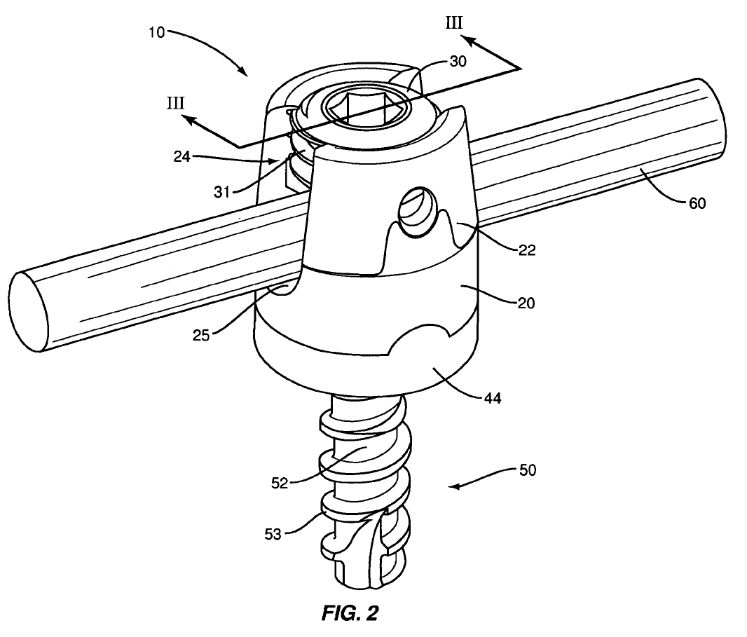

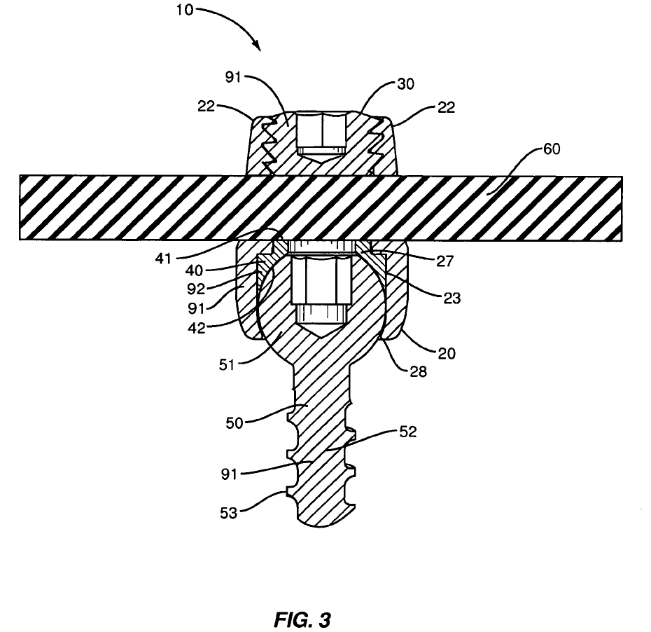

FIG. 2 illustrates the assembly 10 connected with an elongated member 60, and FIG. 3 illustrates a sectional view of the assembly 10 and elongated member 60. The assembly 10 includes the receiver 20 sized to receive the elongated member 60. The set screw 30 attaches to the receiver 20 to capture the elongated member 60. A portion of the anchor 50 fits within a lower section of the receiver 20. The compression member 40 is positioned within the lower section between the anchor 50 and the elongated member 60.

FIG. 4 illustrates the receiver 20 without the other assembly elements and the elongated member 60. Receiver 20 includes a base 21 and opposing sidewalls 22. In one embodiment, the base 20 is generally cylindrical and includes a hollow interior chamber 23 adapted to receive a head 51 of the anchor 50. The hollow interior chamber 23 is sized for the receiver 20 to rotate and pivot about the head 51.

The sidewalls 22 extend from the base 20 and are spaced apart to form a channel 24 sized to receive the elongated member 60. A seating surface 25 may form a lower portion of the channel 24. In one embodiment, the seating surface 25 is curved to substantially match the radius of the elongated member 60 positioned within the channel 24. In one embodiment, the receiver 20 may then be free to rotate and pivot about the head 51 when the elongated member 60 is secured within the channel 24. In another embodiment, the seating surface 25 is positioned such that the elongated member 60 contacts the head 51. For such an embodiment, when the elongated member 60 is secured in the channel 24 it engages the head 51 and locks the position of the receiver 20.

The sidewalls 22 may include threads 26 to receive the set screw 30. Threads 26 may be positioned on the interior of the channel 24 as illustrated in FIGS. 2, 3, and 4, or may be positioned on an exterior of the sidewalls 22 away from the channel 24.

The chamber 23 is positioned in a lower section of the base 21 and is sized to receive the head 51. The chamber 23 includes a central section with a width to accommodate the head 51. Upper and lower constrictions 27, 28 are positioned on each side of the central section to capture the head 51. Each constriction 27, 28 includes a width smaller than the head 51 to maintain the head 50 within the chamber 23. The constrictions 27, 28 may be formed by the receiver 20 itself, or may be formed by additional elements operatively connected to the receiver 20, such as the compression member 40, or a locking ring 75 (FIG. 16).

An exterior surface 29 of the receiver 10 may be generally rounded. Other shapes may also be considered when advantageous for a particular application. For example, the exterior surface 29 may include a flat surface (not shown) to allow a reduced clearance between the receiver 10 and an adjacent receiver 10. A bore 81 may extend through the sidewall 22 and receive a second set screw (not shown) to secure the elongated member 60 within the channel 24.

Set screw 30 attaches to the receiver 20 to capture the elongated member 60 within the channel 24. In one embodiment, the set screw 30 is substantially disc-shaped and is sized to fit within the interior of the channel 24 between the sidewalls 22. Set screw 30 includes exterior threads 31 that engage with the sidewall threads 26. When fully mounted within the channel 24, set screw 30 may apply a compressive force through the elongated member 60 to the head 51 to lock the angular position of the anchor 50 relative to the receiver 20. In another embodiment (not illustrated), set screw 30 is attached to an exterior of the sidewalls 22 and includes a central opening that extends around the receiver 20.

The anchor 50 secures the receiver 20 to a vertebral member. Anchor 50 includes the head 51 and a shaft 52 with helical threads 53 on an outer surface. The head 51 is positioned at an end of the shaft 52 and may include a variety of shapes. Anchor 50 may also be constructed as rivets and pins each with a first end that attaches to the receiver 20, and a second end that attaches to the vertebral members.

The compression member 40 is positioned between the elongated member 60 and head 51. The compression member 40 includes a first side 41 that forms a bearing surface to contact the head 51 and a second side 42 that contacts the elongated member 60. In one embodiment, the second side 42 includes a curved surface that substantially matches the curved shape of the head 51.

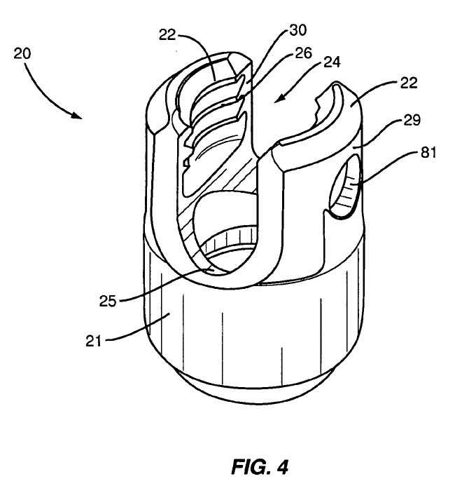

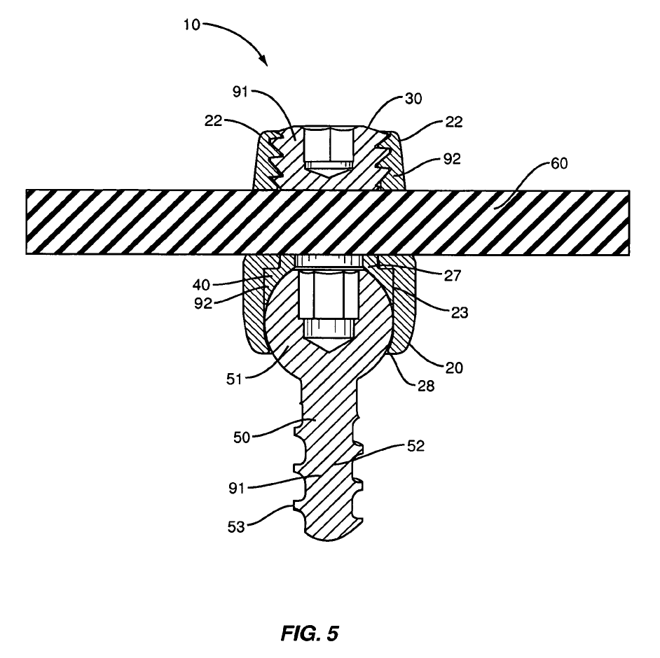

The assembly 10 is formed with at least one of the elements constructed at least in part of a different material than the other elements. FIG. 3 includes an embodiment with the receiver 20, set screw 30 and anchor 50 constructed of the first material 91, and the compression member 40 constructed of the second material 92. The first and second materials 91, 92 include different moduli of elasticity with different resistances to deformation. The placement and usage of the materials 91, 92 are coordinated to optimize the necessary requirements for the assembly 10. FIG. 5 includes another embodiment with the set screw 30 and anchor 50 constructed of the first material 91, and the receiver 20 and compression member 40 constructed of the second material 92. In another embodiment (not illustrated), receiver 20 is constructed of the first material 91, set screw 30 from the second material 92, and the compression member 40 and anchor 50 constructed of a third material.

A variety of different materials may be used for the assembly 10. In one embodiment, the different materials are selected to provide different physical properties to particular elements. In one embodiment, one or more of the elements is constructed of titanium and one or more elements are constructed of cobalt-chrome. In one embodiment, each of the different materials contains less than 1% of nickel.

In another embodiment, at least one of the elements is constructed of stainless steel. It may be desirable for the entire assembly 10 to be constructed of stainless steel, however, stainless steel may exhibit undesirable properties as an implant material. Because stainless steel is relatively heavy and an entire assembly 10 constructed of stainless steel may be burdensome to the patient. Stainless steel also presents problems with magnetic resonance imaging (MRI). Stainless steel is a ferromagnetic material, and elements constructed of stainless steel may be physically moved by the strong magnetic fields produced during an MRI. Stainless steel may also produce artifacts (areas of empty space in the MRI image) around the elements. Additionally, stainless steel elements may increase infection rates, and patients with an allergy to nickel may not tolerate stainless steel receivers. Therefore, a limited number of the elements are constructed of stainless steel to take advantage of the desirable properties, while the other elements are constructed of different materials to reduce the undesirable properties.

The assembly 10 may be constructed of a variety of different materials. Examples include but are not limited to titanium, cobalt chrome, and stainless steel.

The individual elements may also be constructed of two or more different materials. In one embodiment, the receiver 20 includes the base 21 constructed of a first material 91, such as titanium, and the sidewalls 22 constructed of a second material 92, such as cobalt-chrome. The different materials 91, 92 may be necessary because the sidewalls 22 are exposed to forces applied through the elongated member 60 and/or the set screw 30. The forces may cause the sidewalls 22 to splay outward from the channel 24 causing the set screw 30 and the elongated member 60 to loosen or even escape from the receiver 20. Therefore, sidewalls 22 are constructed of the second material 90 to provide greater resistance to these forces.

The different materials are discrete sections that are connected together to form a unitary element. Further, the sections are connected together to form a complete element prior to insertion into the patient. This prevents the sections of the elements from separating while being inserted into the patient.

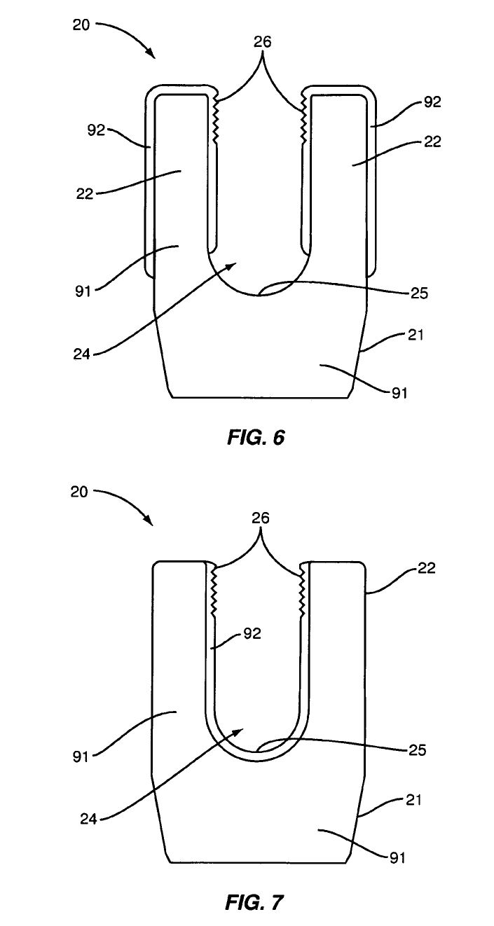

A variety of different methods and structures may be included to connect the sections. FIG. 6 illustrates one embodiment of a receiver 20 with the base 21 formed from a first material 91, and the sidewalls 22 formed by the first material 91 and the second material 92. The second material 92 is positioned on an exterior of the sidewalls 22. Specifically, the second material 92 extends along inner and outer sections of each sidewall 22. The second material 92 extends along the sidewalls 22 and terminates in proximity to the seating surface 25. The inner edges of the second material 92 include the threads 26 that engage with the set screw 30. The second material 92 may extend across the entire width of the sidewalls 22, or a limited width. FIG. 7 illustrates a similar embodiment with the second material 92 connected to one side of the sidewalls 22 and forming the surface of the channel 24.

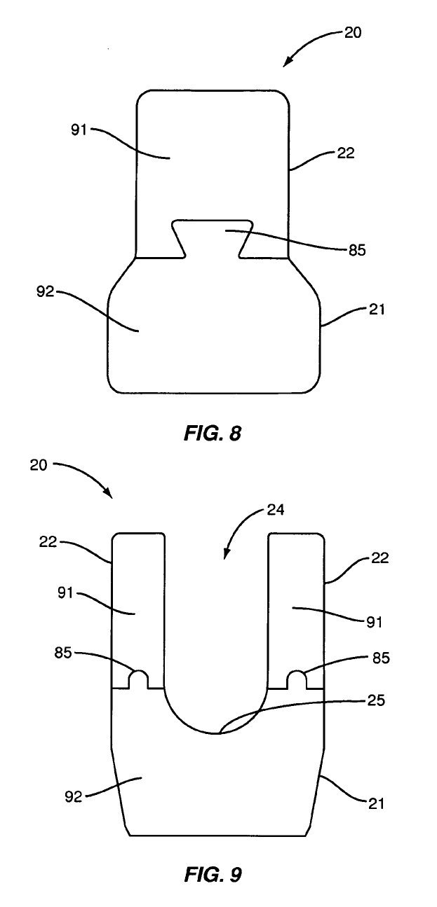

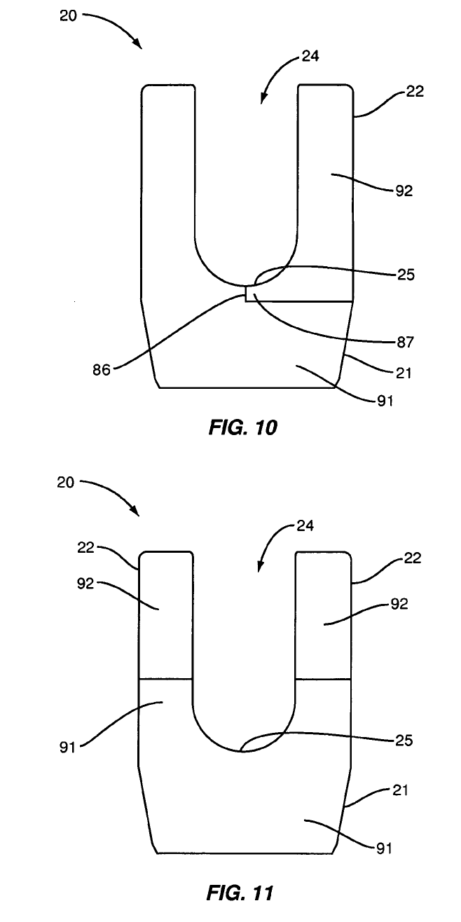

The sections may also include mating surfaces to facilitate the connection between the different materials. FIG. 8 illustrates an embodiment including the sidewalls 22 and the base 21 joined by a joint 85 in the shape of a dovetail. The sidewalls 22 are formed by the first material 91 and the base 21 is formed by the second material 92. FIG. 9 illustrates another embodiment with the sidewalls 22 and base 21 including complementary surfaces that mate together and include joints 85 along complementary surfaces. FIG. 10 includes an embodiment with the base 21 including a recess with a corner 86, and one of the sidewalls 22 including a leg 87 that fits within the corner 86. The base 21 and leg 87 include complementary surfaces that align and form a continuous curve for the seating surface 25. Various other mating surfaces are also contemplated, such as but not limited to tongue and groove, interference fit, welding, and forming.

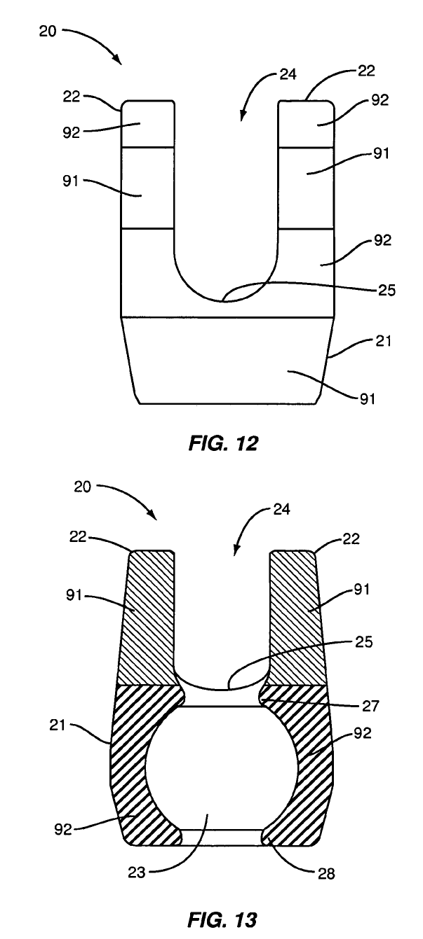

FIG. 11 illustrates an embodiment with the base 21 and lower section of each sidewall 22 formed by a first material 91, and an upper section of each sidewall 22 formed by the second material 92. FIG. 12 illustrates an embodiment with the receiver 20 formed from various vertical levels of materials 91, 92. Both the base 21 and sidewalls 22 are formed from multiple sections of materials 91, 92.

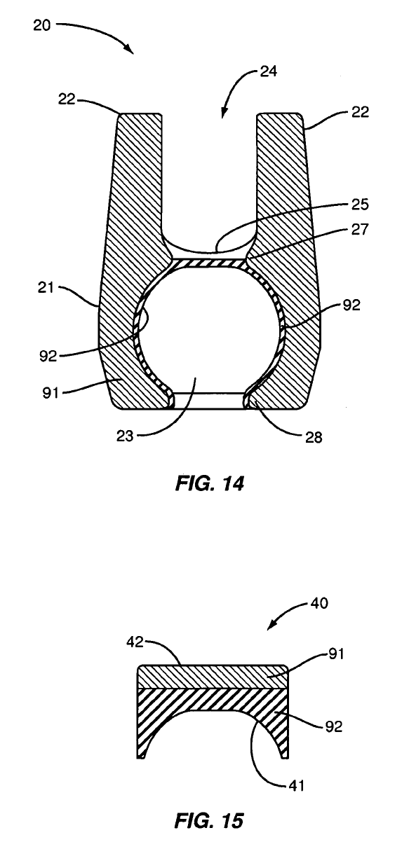

FIGS. 13 and 14 illustrate different embodiments for the chamber 23. FIG. 13 illustrates the lower section of the base 21 including the chamber 23 formed of the second material 92, and the upper section of the base 21 and sidewalls 22 being formed of the first material 91. FIG. 14 illustrates an embodiment with a majority of the receiver 20 formed of the first material 91, and the second material 92 forming an inner surface of the chamber 23.

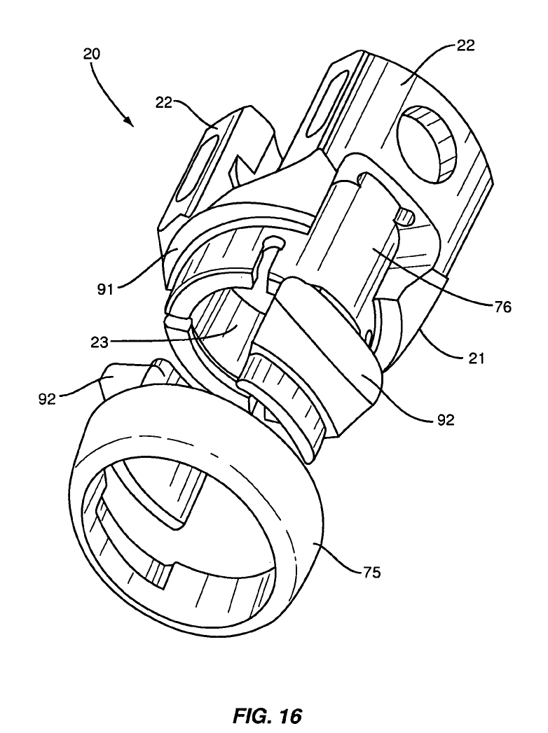

FIG. 15 illustrates an embodiment of the compression member 40 constructed of first and second materials 91, 92. An upper section including the second side 42 is constructed of the first material 91. A lower section including the first side 41 is constructed of the second material 92.

FIG. 16 illustrates an embodiment of a receiver 20 with a first section formed of a first material 91. This first section includes portions of both the base 21 and sidewalls 22. A recess 76 is formed in the first section and extends into a lower section of the sidewalls 22 and the base 21. A second section formed from the second material 92 fits within the recess 76. A locking ring 75 extends over the first and second sections and functions to lock the screw head 51 within the chamber 23. The locking ring 75 may be constructed of the first or second materials 91, 92.

The sections constructed of the different materials may be connected together in a variety of manners. Examples include but are not limited to diffusion bonding, electron beam welding, and biocompatible adhesive. Diffusion bonding is a solid-state joining process capable of joining a wide range of metal combinations. The process may be applied over a variety of durations, applied pressure, bonding temperature, and method of heat application. The bonding is typically formed in the solid phase and may be carried out in vacuum or a protective atmosphere, with heat being applied by radiant, induction, direct or indirect resistance heating. Electron beam welding is a fusion welding process in which a beam of high-velocity electrons is applied to the materials being joined. The sections melt as the kinetic energy of the electrons is transformed into heat upon impact. Pressure is not necessarily applied, though the welding is often done in a vacuum to prevent the dispersion of the electron beam. A biocompatible adhesive is applied to one or both sections and forms a permanent connection. In addition, multiple connection methods may be used on the same sections (e.g., diffusion bonding and biocompatible adhesive).

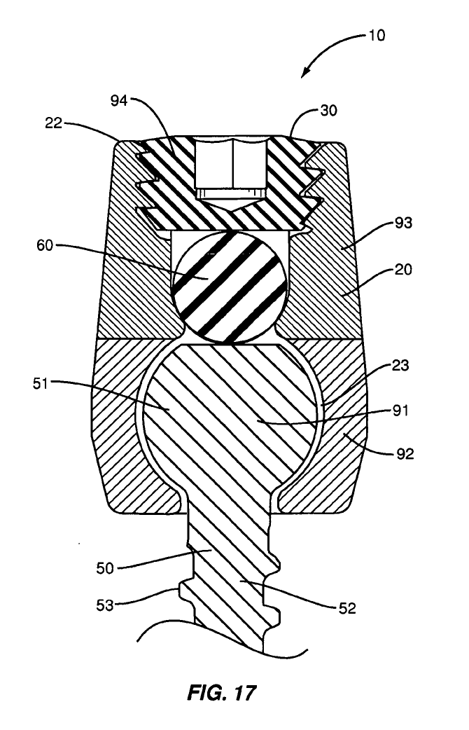

In one embodiment, the assembly 10 includes a compression member 40. In another embodiment as illustrated in FIG. 17, the assembly 10 does not include a compression member 40. The assembly 10 includes a receiver 20, set screw 30, and an anchor 50.

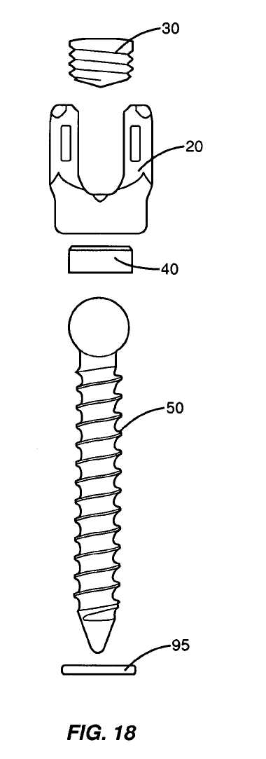

FIG. 18 illustrates another embodiment of a pedicle screw assembly that includes a receiver 20, set screw 30, compression member 40, and bone anchor 50. This assembly further includes a locking ring 95. During assembly, the head of the bone anchor 50 is bottom-loaded into the receiver 20. The locking ring 95 is then moved along the length of the bone anchor 50 and attached to the receiver 20 to capture the head of the bone anchor 50 at least partially within the receiver 20. In one embodiment, the receiver 20 is constructed of cobalt-chrome, with the remaining elements being constructed of titanium. In one embodiment, the locking ring 95 is constructed of cobalt-chrome.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc. and are also not intended to be limiting. Like terms refer to like elements throughout the description.

As used herein, the terms “having”, “containing”, “including”, “comprising”, and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A pedicle screw assembly to position an elongated member within a patient, the assembly comprising:
a receiver including a channel sized to receive the elongated member and a chamber;
an anchor with a head sized to fit within the chamber;
a compression member sized to fit within the chamber and including a first side that contacts against the head and a second side that contacts against the receiver;
at least one of the receiver, anchor, and compression member are constructed of a first material, and at least one of the receiver, anchor, and compression member are constructed of a second material, the first and second materials including different moduli of elasticity such that the second material includes a greater resistance to deformation than the first material.

2. The pedicle screw assembly of claim 1, wherein the receiver and the anchor are constructed of the second material and the compression member is constructed of the first material.

3. The pedicle screw assembly of claim 1, wherein at least one of the receiver, anchor, and compression member are constructed of a third material with the moduli of elasticity different than the first and second materials.

4. The pedicle screw assembly of claim 1, the at least one of the receiver, anchor, and compression member constructed of the first material is also constructed in part from the second material.

5. The pedicle screw assembly of claim 1, wherein the first material includes titanium.

6. The pedicle screw assembly of claim 1, wherein the second material includes cobalt-chrome.

7. A pedicle screw assembly to position an elongated member within a patient, the assembly comprising:
a receiver including a channel sized to receive the elongated member and a chamber;
an anchor with a head sized to fit within the chamber;
the receiver constructed of a first material and the anchor constructed of a second material, the first and second materials including different moduli of elasticity such that one of the anchor and the receiver includes a greater resistance to deformation.

8. The pedicle screw assembly of claim 7, further comprising a compression member sized to fit within the chamber and including a first side that contacts against the head and a second side that contacts against the receiver, the compression member constructed of one of the first and second materials.

9. The pedicle screw assembly of claim 7, wherein one of the receiver and the anchor comprise a first section constructed of the first material and a second section constructed of the second material with the first material being discrete from the second material and the first and second sections forming a non-separable unitary structure prior to being placed within the patient.

10. A pedicle screw assembly to position an elongated member within a patient, the assembly comprising:
a receiver including a channel sized to receive the elongated member and a chamber;
an anchor with a head sized to fit within the chamber;
a compression member sized to fit within the chamber and including a first side that contacts against the head and a second side that contacts against the receiver;
one of the receiver, anchor, and compression member comprising a first section constructed of a first material and a second section constructed of a second material, the first and second materials including different moduli of elasticity such that the second material includes a greater resistance to deformation than the first material with the first material being discrete from the second material;
the first and second sections being constructed as a unitary structure prior to placement within the patient.

11. The pedicle screw assembly of claim 10, wherein the receiver includes the first section constructed of the first material and the second section constructed of the second material, the first section includes a base with the chamber disposed therein, and the second section includes sidewalls that are spaced apart to form the channel.

12. The pedicle screw assembly of claim 10, wherein the second section is constructed exclusively of the second material.

13. The pedicle screw assembly of claim 10, wherein the second section is constructed of a combination of the first and second materials.

14. The pedicle screw assembly of claim 10, wherein each of the first and second materials contains less than 1 percent of nickel.

15. A pedicle screw assembly to position an elongated member within a patient, the assembly comprising:
an anchor including a head and a shaft;
a receiver comprising a base, a pair of sidewalls extending outward from the base and being spaced apart to form a channel sized to receive the elongated member, and a chamber formed in the base and sized to receive the head of the anchor;
the base being constructed of a first material and the sidewalls being constructed of a second material with the first material being discrete from the second material and the base and the sidewalls forming a non-separable unitary structure prior to being placed within the patient;
the first and second materials including different moduli of elasticity to prevent the receiver from deforming due to forces applied through the elongated member.

16. The pedicle screw assembly of claim 15, wherein the sidewalls are constructed exclusively of the second material.

17. The pedicle screw assembly of claim 15, wherein the base and the sidewalls are connected together at a joint.

18. The pedicle screw assembly of claim 15, wherein the base and sidewalls are connected together by diffusion bonding, electron beam welding, and biocompatible adhesive.

19. A pedicle screw assembly to position an elongated member within a patient, the assembly comprising:
an anchor including a head and a shaft;
a receiver comprising a first end including the chamber sized to receive the head of the anchor, and a second end including a channel sized to receive the elongated member;
the first end being constructed of a first material and the second end being constructed of a second material, the second material selected to provide a greater resistance to deformation than the first material from forces applied through the elongated member, the first material being discrete from the second material, and the first and second ends forming a non-separable unitary structure prior to insertion into the patient.

20. The implant of claim 19, wherein the second material includes a higher modulus of elasticity than the first material.

21. The implant of claim 19, wherein the first end is partially constructed of the second material to resist deformation from forces applied through the head of the screw.

22. The implant of claim 19, further comprising a compression member positioned within the chamber with a first side that contacts against the head and a second side that contacts against the elongated member.
